(19) United States
(12) Patent Application Publication
(10) Pub. No.: US 2008/0267919 A1
(43) Pub. Date: Oct. 30, 2008

(54) ANGIOGENESIS-PROMOTING SUBSTRATE

(75) Inventors: Michael Ahlers, Eberbach (DE); Burkhard Schlosshauer, Tuebingen (DE); Lars Dressmann, Kusterdingen (DE)

Correspondence Address: LEYDIG VOIT & MAYER, LTD, 700 THIRTEENTH ST. NW, SUITE 300, WASHINGTON, DC, 20005-3960, US

(73) Assignee: GELITA AG, Eberbach (DE)

(21) Appl. No.: 12/120,390

(22) Filed: May 14, 2008

Related U.S. Application Data
(63) Continuation of application No. PCT/EP2006/010977, filed on Nov. 16, 2006.

(30) Foreign Application Priority Data
Nov. 17, 2005 (DE) 10 2005 054 937.3

(51) Int. Cl. A61K 9/70; A61K 47/42; A61K 35/00

(52) U.S. Cl. 424/93.1; 514/774; 424/444

(57) ABSTRACT

An angiogenesis-promoting substrate which can be manufactured easily and in reproducible quality and which, in particular, under physiological conditions, remains stable for a specified time and yet is biocompatible and resorbable is provided, comprising a porous shaped body formed from a gelatin-containing material which is insoluble and resorbable under physiological conditions.

### CROSS-REFERENCE TO RELATED APPLICATIONS

This patent application is a continuation of PCT Application No. PCT/EP2006/010977, filed Nov. 16, 2006, which claims priority of German patent Application No. 10 2005 054 937.3, filed Nov. 17, 2005, which are each incorporated by reference.

### BACKGROUND OF THE INVENTION

The present invention relates to an angiogenesis-promoting substrate.

In living mammals, endothelial cells which line existing blood vessels form new capillaries wherever these are required. The endothelial cells have the remarkable capability of adapting their number and arrangement to the local requirements. Tissues are dependent upon the blood supply which is provided by the blood vessel system. The vessel system, in turn, is dependent upon the endothelial cells. The endothelial cells create an adaptable life-ensuring system which branches into almost all regions of the body.

While the largest blood vessels, the arteries and veins, have a thick, strong wall of connective tissue and partly smooth muscles and are lined on the inside with only an extremely thin, single layer of endothelial cells, in the finest branches of the vessel system, the capillaries, walls are found which consist solely of endothelial cells and a so-called basal lamina. Endothelial cells thus line the entire blood vessel system running from the heart into the smallest capillary, and they control the passage of materials into and out of the blood stream.

In the event of a deficiency of oxygen, tissue cells release angiogenic factors which activate the growth of new capillaries. Local (mechanical) irritations and infections also cause proliferation of new capillaries, most of which recede and disappear once the inflammation subsides.

The newly forming blood vessels first always develop as capillaries which sprout on existing small vessels. This process is called angiogenesis.

The sprouting of the capillaries propagates until the respective sprout encounters another capillary and can unite with it, so that blood can circulate therein (cf., for example, B. Alberts et al., Molekularbiologie der Zelle, VCH Weinheim, 3^{rd} edition 1995, pages 1360-1364).

Factors which stimulate angiogenesis are widely known and include, for example, the factors HGF, FGF, VEGF and others.

In the literature (cf., for example, EP 1 415 633 A1 and EP 1 555 030 A1), administration of such angiogenesis-stimulating factors in a sustained release matrix was proposed, and a gelatin hydrogel comprising gelatin with an average molecular weight of 100,000 to 200,000 daltons (Da) was recommended as sustained release matrix.

The suitability of various types of collagen as scaffold in the formation of new vessels as well as their anti-angiogenetic effects are described. Reference is made to S. M. Sweeney et al., The Journal of Biological Chemistry, volume 278, No. 33, pages 30516 to 30524 (2003) and to R. Xu et al. in Biochemical and Biophysical Research Communications 289, pages 264 to 268 (2001) as examples of this literature.

### BRIEF SUMMARY OF THE INVENTION

The object underlying the present invention is to provide an angiogenesis-promoting substrate which can be manufactured easily and in reproducible quality and which, in particular, under physiological conditions, remains stable for a specified time and yet is biocompatible and resorbable.

This object is accomplished by an angiogenesis-promoting substrate comprising a porous shaped body formed from a gelatin-containing material which is insoluble and resorbable under physiological conditions.

### DETAILED DESCRIPTION OF THE INVENTION

Surprisingly, it has been found that porous shaped bodies made from a gelatin-containing material which is insoluble and resorbable under physiological conditions have a very pronounced angiogenesis-promoting effect in that, in particular, the angiogenesis causes formation of blood vessels in a considerable density within the porous shaped body, so that targeted angiogenesis is possible by placing the porous shaped bodies at the desired locations on the body of the patient or animal to be treated.

In particular, it is surprising that the gelatin-containing material processed as porous shaped body as such acts as angiogenesis-promoting without, as is otherwise described in the literature, angiogenesis-promoting factors such as, for example, the aforementioned factors VEGF, FGF, HGF and others, being required.

The gelatin-containing material is preferably a gelatin-based material and consists predominantly of gelatin. This means that the gelatin constitutes the largest proportion where other components are used in the material.

Further preferred is use of a gelatin-based material consisting substantially entirely of gelatin.

Particularly suitable gelatin types are pigskin gelatin, which is preferably high-molecular and has a Bloom value of approximately 160 to approximately 300 g.

To a considerably lesser extent, an angiogenesis-stimulating effect is observed with low-molecular, water-soluble gelatin having an average molecular value of less than 6 kDa, but such an effect is comparatively unspecific when compared with other agents that likewise stimulate to a lesser extent.

Therefore, the gelatin used preferably has an average molecular weight greater than 6 kDa.

To ensure optimum biocompatibility of the substrate according to the invention in medical use, a gelatin having a particularly low content of endotoxins is preferably used as starting material. Endotoxins are products of metabolism or fractions of microorganisms which occur in the raw animal material. The endotoxin content of gelatin is indicated in international units per gram (I.U./g) and determined in accordance with the LAL test, the performance of which is described in the fourth edition of the European Pharmacopoeia (Ph. Eur. 4).

To keep the content of endotoxins as low as possible, it is advantageous to kill the microorganisms as early as possible in the course of the gelatin production. Furthermore, appropriate hygiene standards should be maintained during the manufacturing process.

The endotoxin content of gelatin can thus be drastically lowered by certain measures during the manufacturing process. These measures primarily include the use of fresh raw materials (for example, pigskin) with avoidance of storage times, thorough cleaning of the entire production plant immediately before start of the gelatin production and possibly exchange of ion exchangers and filter systems in the production plant.

The gelatin used within the scope of the present invention preferably has an endotoxin content of 1,200 I.U./g or less, even more preferred 200 I.U./g or less. Optimally, the endotoxin content lies at 50 I.U./g or less, determined, in each case, in accordance with the LAL test. In comparison with this, many commercially available gelatins have endotoxin contents of over 20,000 I.U./g.

Since gelatin dissolves rapidly under the physiological conditions to which the substrate is exposed when used for promoting angiogenesis and the porous shaped body would, therefore, quickly lose its structural integrity, the gelatin-containing material is preferably used with a specified degree of cross-linking.

In accordance with a further embodiment of the present invention, this can be counteracted by using the gelatin together with another component which dissolves slower (examples of such resorbable biopolymers are chitosan and hyaluronic acid). Such components may be used for the purpose of temporary immobilization of the gelatin proportions.

If cross-linking is chosen for stabilization of the material, then, in particular, the gelatin proportion of the gelatin-containing material can be cross-linked, and chemical cross-linking, or also enzymatic cross-linking can be resorted to.

Preferred chemical cross-linking agents are aldehydes, dialdehydes, isocyanates, carbodiimides and alkyl dihalides. Formaldehyde, which simultaneously effects a sterilization of the shaped body, is particularly preferred.

The enzyme transglutaminase, which effects a linking of glutamine and lysine side chains of proteins, in particular, also of gelatin, is preferred as enzymatic cross-linking agent.

The stability with respect to resorption under the physiological conditions referred to hereinabove, to which the material is exposed during its use, can be simulated under corresponding standard physiological conditions in vitro.

Here a PBS buffer (pH 7.2) is used at 37 C, and under these conditions the substrates can be tested and compared as to their time-dependent stability behavior.

The structure of the porous shaped body is preferably stabilized by a two-stage cross-linking, where at a first stage the gelatin-containing material in solution is subjected to a first cross-linking reaction, the material is then foamed, and a porous shaped body obtained therefrom is then further cross-linked at a second cross-linking stage.

Whereas at the first cross-linking stage, the cross-linking takes place in solution, two different cross-linking processes are possible for the second cross-linking stage.

The porous shaped body can be brought into contact with a cross-linking solution and the degree of cross-linking thus further increased, or, in particular, when the gelatin itself is cross-linked, the porous shaped body can be exposed to a formaldehyde vapor, so that the formaldehyde components penetrating through the porous shaped body via the gaseous phase lead to a further cross-linking.

The two-stage cross-linking has, in particular, the advantage that overall a higher degree of cross-linking is obtainable, which, in addition, is then achievable substantially uniformly over the entire cross-section of the porous shaped body. As a consequence of this, the degradation characteristics of the porous shaped body during the resorption are homogenous, so that it substantially maintains its structural integrity for the intended period of time in dependence upon the degree of cross-linking and is then completely resorbed in a relatively short time, whereby the structural integrity is lost.

In view of the above-explained effect of the concentration of the angiogenesis on the area taken up by the porous shaped body itself, this has the great advantage that the angiogenesis can be controlled very well and focused on the desired places by the attending physician.

Depending on the application, the resorption stability of the shaped body can, in turn, be adjusted via the variation of the degree of cross-linking, and, consequently, the point in time at which the porous shaped body loses its structural integrity specified in an application-oriented manner.

For many applications, the degree of cross-linking should be so selected that under the standard physiological conditions mentioned hereinabove 20 wt % or less of the gelatin-containing material is degraded over 7 days.

The porous shaped body can be made with very different structures, which have not yet been discussed.

In a preferred embodiment of the invention, the shaped body of the substrate has a fiber structure. This fiber structure may have a woven or knitted structure. Alternatively, a fiber structure in the form of a fleece is also possible.

A completely different structure of the shaped body of the substrate according to the invention resides in the sponge structure, which preferably has a proportion of open pores. Further preferred is a sponge structure with a substantially open-pored structure.

Common to all embodiments of the porous shaped body is that the porosity makes it possible for the endothelial cells to migrate into the substrate and penetrate it. By virtue of its porosity, the shaped body also enables the endothelial cells to form capillary vessels extending into the substrate.

Where the sponge structure is selected for the porous shaped body, it is advantageous for it to have a multiplicity of pores with an average pore size ranging from approximately 50 to 500 μm.

The porosity of the other porous shaped bodies should be so selected that similar pore structures exist there, as these are optimally suited for receiving the endothelial cells and allowing capillary vessels to grow through the substrate.

The porous shaped body of the angiogenesis-promoting substrates according to the invention has the additional advantage that one or more pharmaceutically active agents not based on gelatin can be incorporated in the pores of the shaped body.

Moreover, the pores of the shaped body can already be colonized with cells before the substrate is placed at the location of the human or animal body to be treated.

The geometrical shape of the substrate has so far not been discussed in detail, however, it will be understood that the substrate may be selected so as to vary widely in its outer dimensions. In many applications, a sheet substrate for promoting the blood vessel formation can be used with advantage as implant. In addition, the substrate may, however, also be in the form of small particles, in particular, in powder form, the particles of the powder preferably being produced, in particular, by grinding, from a sponge structure, a fleece, a knitted or a woven structure.

### BRIEF DESCRIPTION OF THE SEVERAL VIEWS OF THE DRAWINGS

These and further advantages of the invention are explained in greater detail hereinbelow with reference to the drawings and the examples. There are shown in:

FIG. 1 the degradation behavior of various angiogenesis-promoting substrates according to the invention;

FIGS. 2a and 2b a schematic representation of the trial set-up for examining the angiogenesis by means of a chorioallantoic membrane (CAM);

FIG. 3 the angiogenesis induced by an angiogenesis-promoting substrate according to the invention on a CAM after 3, 5 and 7 days;

FIG. 4 a diagram showing the formation of blood vessels around the angiogenesis-promoting substrate;

FIG. 5 a diagram showing the development of blood vessels in the angiogenesis-promoting substrate itself;

FIG. 6 three light-microscopic images of a reference substrate consisting of collagen sponge after 2, 5 and 7 days; and

FIGS. 7a and 7b four light-microscopic images of an angiogenesis-promoting substrate according to the invention after 3, 5, 7 and 8 days.

### EXAMPLES

### Example 1

### Production and Characteristics of Shaped Bodies with a Cell Structure Based on Cross-Linked Gelatin

Five batches of a 12 wt % solution of pigskin gelatin (Bloom strength 300 g, average molecular weight 140 kDa) in water were produced by dissolving the gelatin at 60° C., degassed with ultrasound, and each mixed with the corresponding amount of an aqueous formaldehyde solution (1.0 wt %, room temperature), which resulted in 1500 ppm formaldehyde (based on the gelatin). No formaldehyde was added to a sixth batch.

The homogenized mixtures were tempered to 45° and after a reaction time of 10 min were mechanically foamed with air. The approximately 30-minute foaming operation was performed on the six batches with a different ratio of air to gelatin solution, which resulted in cell structures with different wet densities and pore sizes in accordance with Table 1.

The foamed gelatin solutions, exhibiting a temperature of 26.5° C., were poured into molds having dimensions of 40×20×6 cm and dried for approximately four days at 26° C. and a relative air humidity of 10%.

The dried shaped bodies of all six batches exhibit a sponge-like cell structure (referred to hereinbelow as sponges). They were cut into 2-mm-thick layers and for the second cross-linking step were exposed for 17 hours in a desiccator to the equilibrium vapor pressure of a 17 wt % aqueous formaldehyde solution at room temperature. For the sixth batch this was the first (and only) cross-linking step. To achieve a uniform gassing of the entire volume of the shaped bodies, the desiccator was evacuated and aerated again two to three times.

The pore structure of the sponges was determined light-microscopically and was confirmed by scanning electron microscopy.

TABLE 1

| | Wet density | Dry density | Average pore size |
|---|---|---|---|
| Batch | (mg/cm³) | (mg/cm³) | (μm) |
| 1-1 | 100 | 20 | 250 |
| 1-2 | 175 | 27 | 200 |
| 1-3 | 300 | 50 | 125 |
| 1-4 | 530 | 70 | 100 |
| 1-5 | 600 | 100 | 75 |
| 1-6 | 78 | 12 | 300 |

To determine the stability of the sponges, 30×30×2 mm-large pieces were weighed, each placed in 75 ml PBS buffer and stored at 37° C. Following the respective storage time, the pieces were washed for 30 min in water, dried and weighed.

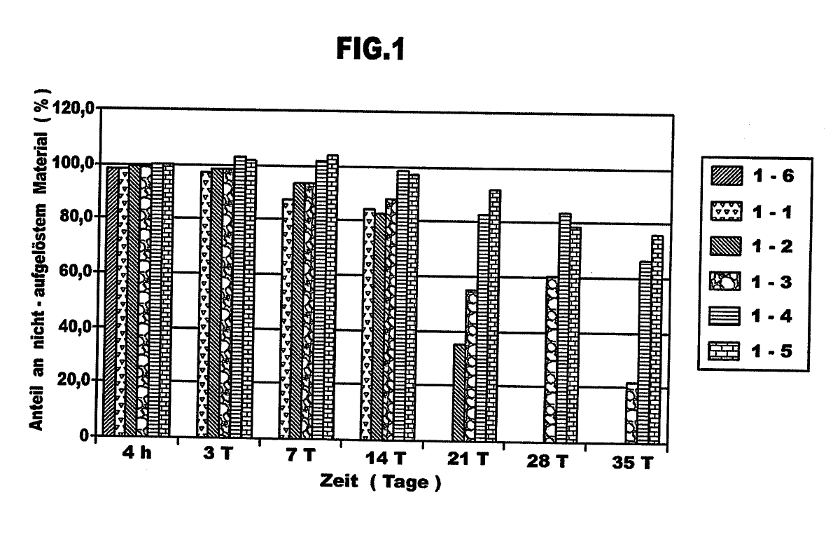

FIG. 1 shows the disintegration, i.e., resorption behavior of the sponges 1-1 to 1-5 cross-linked at two stages and of the sponge 1-6 cross-linked once (the sequence of the bars shown is: 1-6, 1-1, 1-2, 1-3, 1-4, 1-5, respectively).

Whereas sponge 1-6 had already completely disintegrated after three days, all sponges cross-linked at two stages were still conserved to more than 80% even after 14 days. Considerable differences are, however, evident in the further degradation behavior, which are due to the different foaming densities of the materials. Sponge 1-1 is completely disintegrated after 21 days and sponge 1-2 after 28, while sponges 1-4 and 1-5 are still substantially maintained even after 35 days. This offers a further possibility of specifically influencing the degradation behavior of these sponges or cell structure materials independently of other parameters.

The characteristics of the cell structure materials can, however, also be significantly modified by way of a change in the gelatin concentration in the starting solution. Higher gelatin concentrations result in broader (thicker) cell walls or webs between the individual pores, which is reflected in an increased breaking resistance of the corresponding sponges.

The breaking resistance increases continuously as the gelatin concentration in the starting solution is increased from 10 to 18 wt %, and a wide range of approximately 500 to almost 2,000 newtons is covered. At the same time, the deformation up to breakage changes only slightly. Surprisingly, the correlation between breaking force and gelatin concentration is substantially independent of the degree of cross-linking.

By way of the degree of cross-linking, i.e., by the choice of the concentration of the cross-linking agent, on the other hand, the stability of the shaped bodies, in particular, in view of proteolytic disintegration, can be influenced.

### Example 2

Samples having the dimensions 15×15×2 mm were produced from shaped bodies obtainable in analogy with Example 1 and cross-linked twice (dry density 22 mg/ml, average pore size approximately 250 μm). These are referred to hereinbelow as implants.

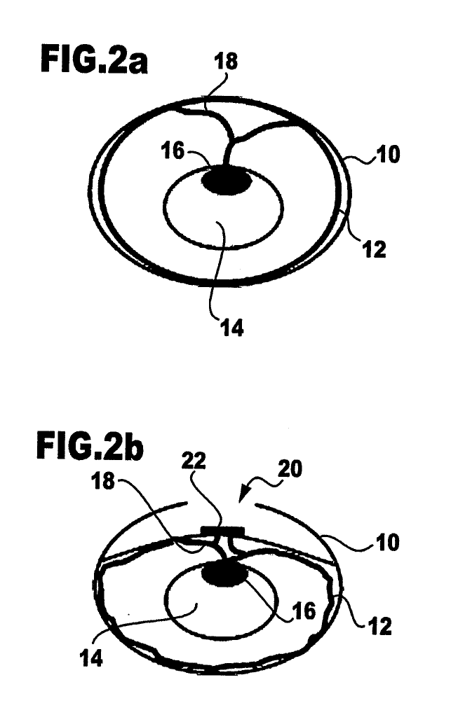

The angiogenesis-promoting characteristics of these implants were examined in a trial on fertilized hen's eggs, which is represented schematically in FIG. 2.

FIG. 2a shows schematically the structure of a hen's egg in cross section. The chorioallantoic membrane 12 (referred to hereinbelow as CAM for short) is located beneath the lime shell 10. Starting from the embryo 16 located at the edge of the egg yolk 14, a formation of extra-embryonic blood vessels 18 occurs, which propagate along the CAM. If part of the egg white is removed with a cannula, a window 20 can subsequently be cut in the lime shell 10 without injuring the CAM 12 (as shown in FIG. 2b). An implant 22 can now be placed on the CAM 12 and its effect on the blood vessel formation examined (cf., for example, J. Borges et al. (2004) Der Chirurg 75, 284-290).

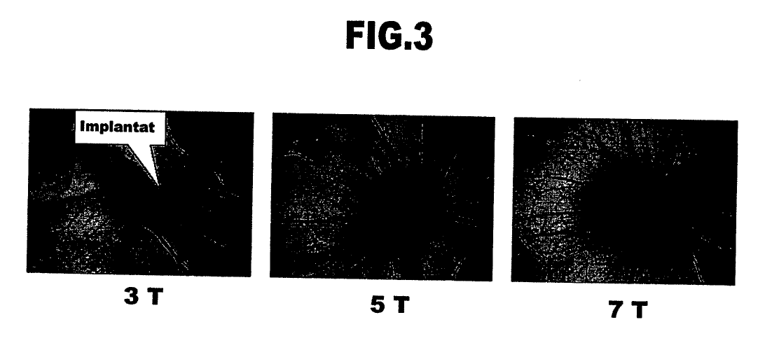

FIG. 3 shows the reorientation and new formation of blood vessels in light-microscopic images after 3, 5 and 7 days.

In addition to the substrate according to the invention, comparable sponge-like materials made of collagen (renatured, bovine collagen, density 5.6 mg/cm³, obtainable from the company Innocoll) and poly-DL-lactide (manufacturer ITV Denkendorf) were tested as reference examples.

All implants were placed on a CAM and after 3, 4, 5, 6 and 7 days the number of blood vessels which had developed in the direct vicinity of the implants was determined. As is apparent from FIG. 3, within a few days the blood vessels clearly orientate themselves towards the angiogenesis-promoting substrate or the reference samples made of sponge-like collagen and poly-DL-lactide.

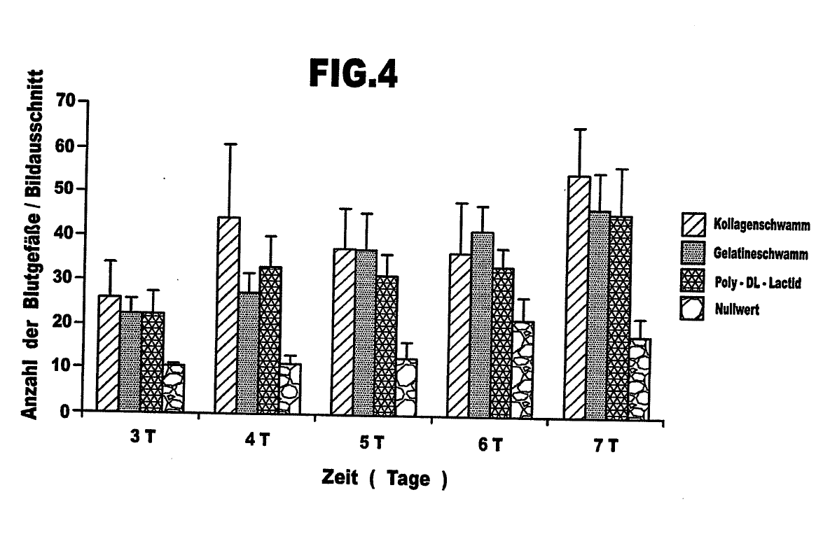

The evaluation according to the number of blood vessels per image detail around the substrate is represented in FIG. 4. It is evident that a distinctly higher number of blood vessels is present in all three samples in comparison with the zero value (CAM without an implant placed thereon), and similar effects, in particular, seen in relation to the zero value, were achieved for all three samples.

This means that all tested materials lie with respect to their angiogenetic effect in their environment at approximately the same increased level. The observed effect is caused over a certain distance and is, therefore, presumably due to so-called diffusible factors.

The CAM is a tissue which represents the interface between air and egg liquid. Merely the mechanical stimulation caused by placing the substrate on the CAM possibly activates receptors, which could lead to a release of pro-angiogenetic factors such as, for example, VEGF, of the cells. Endothelial cells could thereby be attracted, and formation of blood vessels directed at the implant would then take place.

Another possible explanation is that atmospheric oxygen is hindered in reaching the epithelial tissue by the placement of the implant. A so-called anoxia thus occurs in the region of the implant, as less oxygen is available in the epithelial tissue. The typical reaction of cells to anoxia is to release VEGF, whereby reformation or new formation of blood vessels is induced. This means that the inadequately supplied part of the cells organizes new supply lines for themselves. This biological phenomenon presumably occurs above a critically undersupplied (deformed) tissue area.

This would explain why in trials where narrow rubber rings were merely placed on the CAM (very small occupied surface area), no pro-angiogenetic effects were observed.

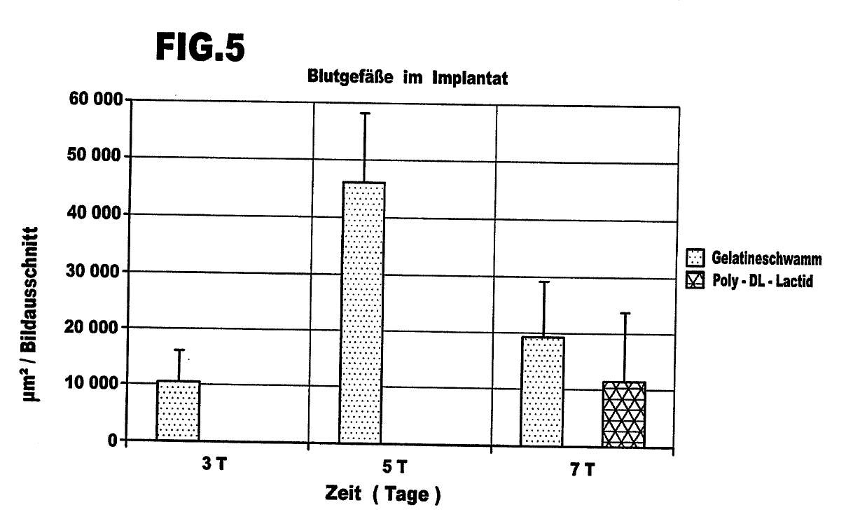

The area of the blood vessels (in μm²) within the substrates or implants of the comparative materials and the angiogenesis-promoting substrate of the present invention after 3, 5 and 7 days is indicated in FIG. 5. In the sequence of columns depicted, the order is gelatin sample, collagen sample, poly-DL-lactide sample.

As is apparent from FIG. 5, only in the case of the angiogenesis-promoting substrate according to the invention is a measurable amount of blood vessels evident in the implant itself after 3 days, whereas no measurable amounts of blood vessels are present in the collagen sponge and the poly-DL-lactide sponge.

After 5 days, an extreme increase in the measurable blood vessels is evident in the angiogenesis-promoting substrates according to the invention, whereas still no effect whatever is observed for the poly-DL-lactide sample or for the collagen sponge.

After 7 days, the amount of blood vessels in the implant in the case of the angiogenesis-promoting substrate according to the invention decreases significantly, but the effect is still about twice as high as after 3 days. At this point in time, there is still no evidence of measurable results in the collagen sponge, whereas in the poly-DL-lactide sponge an effect similar to that already ascertained after 3 days in the gelatin sponge implant sample according to the invention now appears.

To evaluate the samples and determine the number of blood vessels in the implant, frozen sections were produced from the respective samples and stained with DAPI, in order to analyze the area of the blood vessels within the implant. To do so, images were made from the central region of the sections and then quantitatively evaluated by image-processing techniques. In the case of collagen sponges, no blood vessel formation was observed in the central region. In the case of the poly-DL-lactide sponges, angiogenesis was only to be ascertained after 7 days, accompanied by a progressive connective tissue cell colonization. All in all, however, the colonization with cells proceeded significantly slower in the case of this comparative sample, too, than in the implants according to the invention.

The reduction of the blood vessels in the implant according to the invention after 7 days is expressed in a decrease in the measured area. This could be due to the blood vessel network being reduced again to that extent which is actually required for the implant regions owing, for example, to relatively few other cell types that need to be supplied having meanwhile immigrated. This corresponds to a phenomenon which also occurs with infections where a blood vessel network recedes again once the inflammation diminishes.

A comparison of the data obtained with the angiogenesis-promoting substrates according to the invention reveals that use of a porous shaped body made of a gelatin-containing material which is insoluble and resorbable under physiological conditions is of considerable importance.

### Example 3

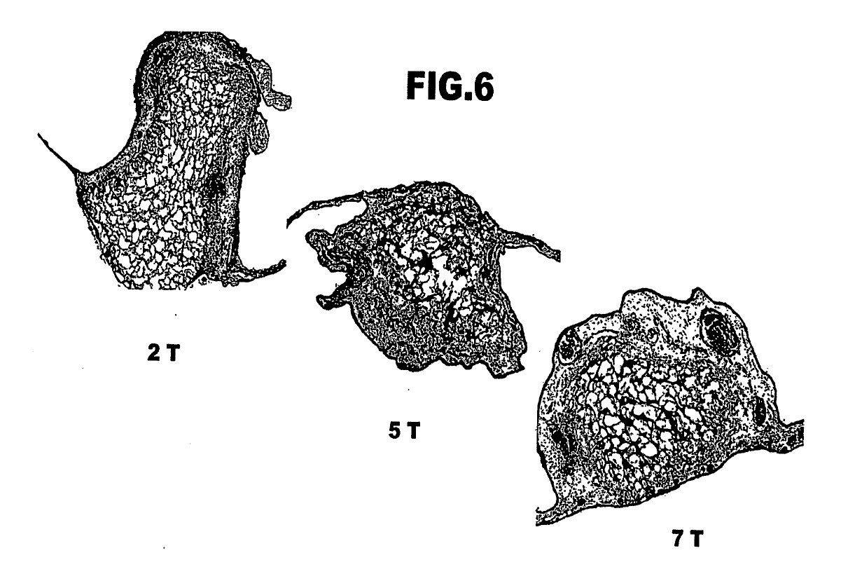

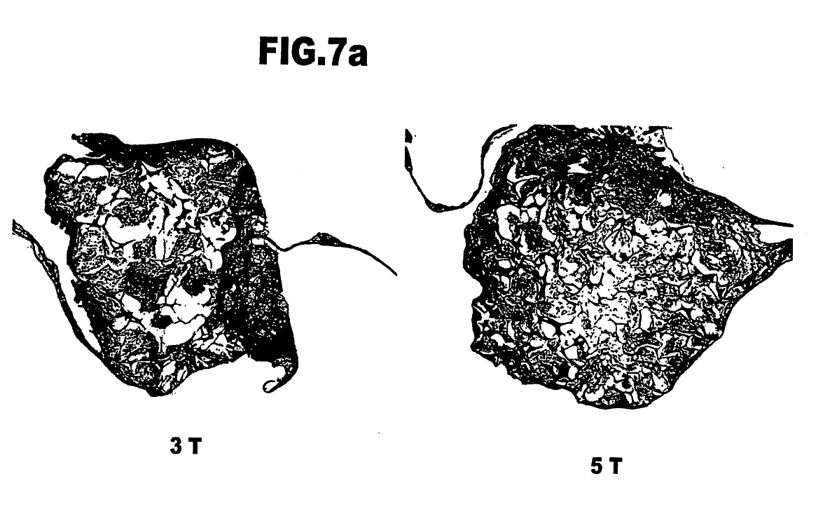

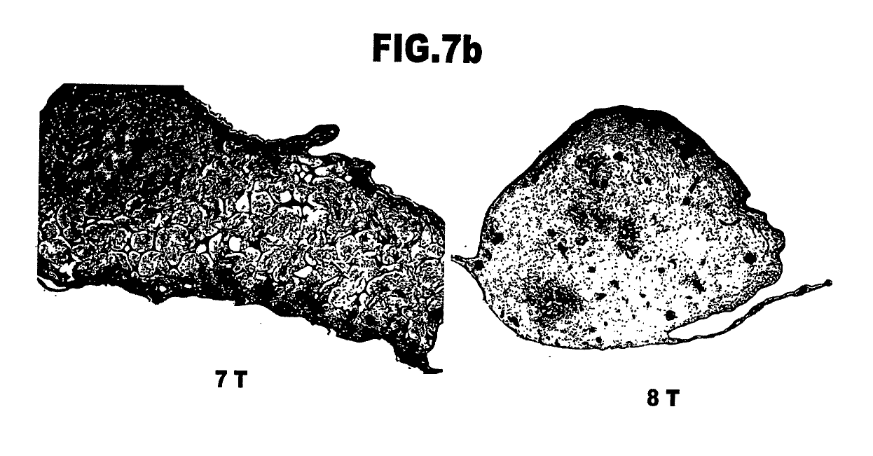

To elucidate the effect according to the invention, the development of the angiogenesis in collagen sponge and gelatin sponge is shown in light-microscopic images in FIGS. 6 and 7.

Whereas in the case of the collagen sponge angiogenesis takes place only in the environment of the sample, and few to even no capillary vessels are to be observed in the sample itself even after 7 days (FIG. 6), in contrast to this, in the case of the gelatin sponge sample, growth throughout the entire substrate is to be observed as the trial time advances (FIGS. 7a and b). These light-microscopic images, too, again provide evidence of the importance of the presence of gelatin in the porous shaped body.

Solutions containing angiogenetic factors can also be included in the porous shaped body and the pro-angiogenetic effects thus further promoted at least in the initial phase.

Furthermore, it appears possible to use the porous shaped body as carrier for pharmaceutically active agents without its effect of promoting angiogenesis being thereby inhibited.

1. An angiogenesis-promoting substrate, comprising a porous shaped body made of a gelatin-containing material which is insoluble and resorbable under physiological conditions.

2. (canceled)

3. The substrate in accordance with claim 2, wherein the gelatin-based material consists substantially entirely of gelatin.

4. The substrate in accordance with claim 1, wherein the gelatin comprises high-molecular gelatin.

5. The substrate in accordance with claim 4, wherein the high-molecular gelatin has a Bloom value ranging from approximately 160 to approximately 300 g.

6. The substrate in accordance with claim 4, wherein the gelatin has an average molecular weight greater than 6 kDA.

7. The substrate in accordance with claim 4, wherein the gelatin has an endotoxin content, determined in accordance with the LAL test, of 1,200 I.U./g or less.

8. The substrate in accordance with claim 1, wherein the gelatin-containing material has a specified degree of cross-linking.

9. (canceled)

10. The substrate in accordance with claim 8, wherein the degree of cross-linking is so selected that under standard physiological conditions 20 wt % or less of the gelatin-containing material is degraded over 7 days.

11. The substrate in accordance with claim 8, wherein the gelatin-containing material is cross-linked using formaldehyde.

12. The substrate in accordance with claim 8, wherein the gelatin-containing material is cross-linked enzymatically.

13. The substrate in accordance with claim 1, wherein the shaped body has a fiber structure.

14. The substrate in accordance with claim 13, wherein the fiber structure comprises a woven or knitted fabric.

15. The substrate in accordance with claim 13, wherein the fiber structure comprises a fleece.

16. The substrate in accordance with claim 1, wherein the shaped body comprises a sponge structure.

17. (canceled)

18. The substrate in accordance with claim 16, wherein the sponge structure is a substantially open-pored structure.

19. The substrate in accordance with claim 16, wherein the sponge structure has a multiplicity of pores with an average pore size of from approximately 50 to 500 μm.

20. The substrate in accordance with claim 1, further comprising gelatin having an angiogenetic effect incorporated into the pores of the shaped body.

21. The substrate in accordance with claim 20, wherein the gelatin having an angiogenetic effect is incorporated into the shaped body so as to be releasable with delay.

22. The substrate in accordance with claim 1, further comprising one or more pharmaceutically active agents not based on gelatin incorporated in the pores of the shaped body.

23. The substrate in accordance with claim 1, further comprising cells colonizing the pores of the shaped body.

24. The substrate in accordance with claim 1, wherein the shaped body is a sheet material.

25. The substrate in accordance with claim 1, wherein the shaped body is present as particles in powder form.

26. The substrate in accordance with claim 25, wherein the powder particles are produced by grinding a porous, sponge-like or fiber material.

27. (canceled)

28. (canceled)

29. The substrate according to claim 7, wherein the gelatin has an endotoxin content, determined in accordance with the LAL test of 200 I.U./g or less.
